(19) United States
(12) Patent Application Publication
(10) Pub. No.: US 2016/0251367 A1
(43) Pub. Date: Sep. 1, 2016

(54) SYNTHESIS OF TRITOQUALINE SALTS

(71) Applicant: Chrysalis Pharma, Montceau Les Mines (FR)

(72) Inventors: Gaetan TERRASSE, Montceau les Mines (FR); Vincent COTEREAU, SURESNES (FR); Catherine BUR, Orgerus (FR)

(21) Appl. No.: 14/940,475

(22) Filed: Nov. 13, 2015

Related U.S. Application Data
(60) Provisional application No. 62/079,046, filed on Nov. 13, 2014.

(51) Int. Cl. C07D 491/056; C07C 309/04; C07C 309/19; C07C 53/10; C07C 57/15

(52) U.S. Cl. CPC C07D 491/056; C07C 53/10; C07C 57/15; C07C 309/19; C07C 309/04; C07C 2102/42

(57) ABSTRACT

A formulation of tritoqualine salt having the purpose of improving the solubility of these novel compounds and having the following general structure:

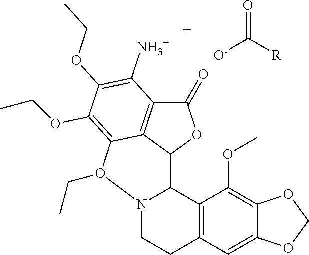

Various publications are referenced throughout this application. The descriptions of these publications in their entirety are incorporated by reference into this application to describe herein the prior art to which this invention belongs.

### FIELDS OF THE INVENTION

The invention describes the synthesis of tritoqualine salts and also the use thereof.

### PRIOR ART OF THE INVENTION

7-Amino-4,5,6-triethoxy-3-(5,6,7,8-tetrahydro-4-methoxy-6-methyl-1,3-dioxolo[4,5-g]isoquinolin-5-yl) phthalide or tritoqualine is a medicament which has been marketed in Europe in the form of 100 mg tablets for treating allergies.

The molecule is highly insoluble and this therefore reduces its bioavailability. Hitherto, no tritoqualine salt has been described in the scientific literature. However, several patents describe the possibility of using tritoqualine salts, but without demonstrating the feasibility thereof (patent EP2170317 G. Terrasse et al. and also the patents derived from this main patent WO 2008100539A1).

Due to its insolubility in water and bodily fluids, the use of tritoqualine is greatly limited.

Inhaled forms (nasal or respiratory) or eye lotion forms appear virtually impossible to produce, due to this insolubility.

The only forms of tritoqualine that are known are oral forms in the form of tablets. A brief reminder of what salts are is given hereinbelow:

In chemistry, a salt is an ionic compound composed of cations and anions forming a neutral product with zero net charge. The anion originates from the acid, which is its conjugate base; the cation originates from the base, which is its conjugate acid. These ions may be either mineral (chloride Cl⁻) or organic (acetate CH₃—COO⁻) and either monoatomic (fluoride F⁻) or polyatomic (sulfate SO₄²⁻).

Salts are generally solid crystals with a relatively high melting point, but salts with low melting points exist. Their solubility in a solvent depends on the charge and on the size of the ions, on the dielectric constant of the solvent and many other factors.

Salts may be clear and transparent (sodium chloride), opaque, and even metallic and lustrous (iron disulfide). The color of the crystals or of the saline solutions depends on the ions which constitute the salt. Salts are colorless when their ions are themselves colorless.

The aim of the invention described herein is to produce a physiological salt of tritoqualine, allowing its solubility to be improved.

The additional constraint is to obtain a crystalline form that is readily usable in a pharmaceutical form.

The salt must also dissociate easily in bodily fluids in order for the tritoqualine to be able to act as a medicament.

In addition, the salts must not be toxic, and their dissolution must not give toxic compounds.

### SUMMARY OF THE INVENTION

The invention describes the racemic tritoqualine salts. The composition of racemic tritoqualine comprises the RR and SS enantiomers.

The object of the invention is to make tritoqualine more soluble in salt form.

In its base form, tritoqualine has a solubility of 0.5 mM/L.

The synthesis of several tritoqualine salts shows that several of them increase the solubility considerably and surprisingly.

The inventors synthesized tritoqualine salts with the following acids:

ethanoic acid, adipic acid, hexanedioic acid, o-aminobenzoic acid, anthranilic acid, m-aminobenzoic acid, para-aminobenzoic acid, glycine, ascorbic acid, aspartic acid, benzoic acid, bromoethanoic acid, o-bromobenzoic acid, m-bromobenzoic acid, citric acid, lactic acid, 2-hydroxypropanoic acid, maleic acid, z-butenedioic acid, malonic acid, propanedioic acid.

The mesylate, camphorsulfonic acid, sulfate and hydrochloride salts were also synthesized.

Most of these salts substantially increase the solubility of tritoqualine, in a proportion of from 10 to 50 000.

A general synthetic method was developed according to the following mode:

Tritoqualine (the base) was placed in equimolar contact with the acids mentioned above in a suitable solvent.

The following solvents were used:

acetone, 2-butanone, absolute ethanol, ethyl acetate, n-heptane, isopropyl acetate, methanol, 4-methyl-2-pentanone, propanol, tetrahydrofuran, toluene.

The mixture was stirred in a rotary evaporator with a vacuum regulator allowing the mixture to be concentrated to the point of crystallization of the salts, the temperature ranging from 0° to more than 50° depending on the salts and the solvents.

### BRIEF DESCRIPTION OF THE FIGURES

FIG. 1: represents the general formula of tritoqualine (7-amino-4,5,6-triethoxy-3-(5,6,7,8- tetrahydro-4-methoxy-6-methyl-1,3-dioxolo[4,5-g]isoquinolin-5-yl) phthalide.

FIG. 2: illustrates the general form of a tritoqualine salt.

FIG. 3: illustrates the NMR of tritoqualine

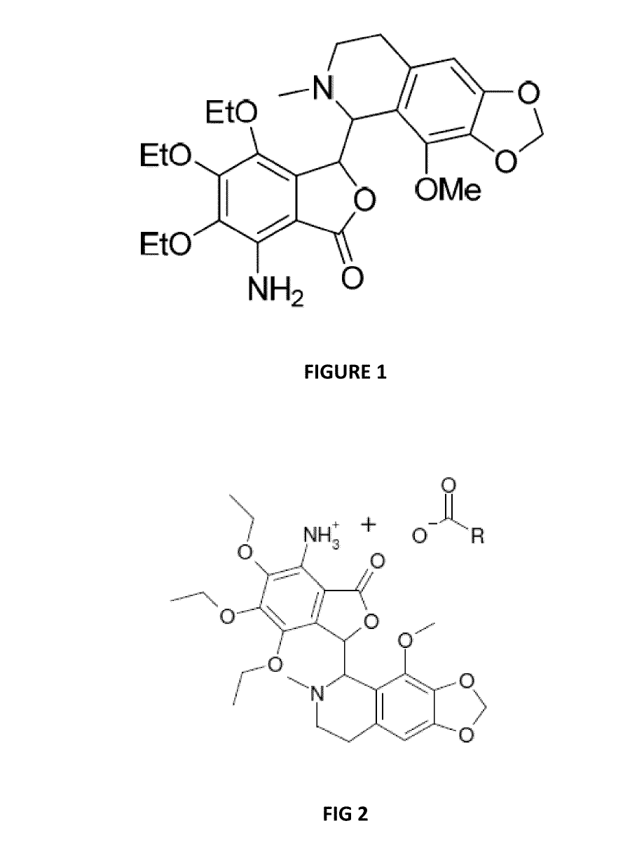

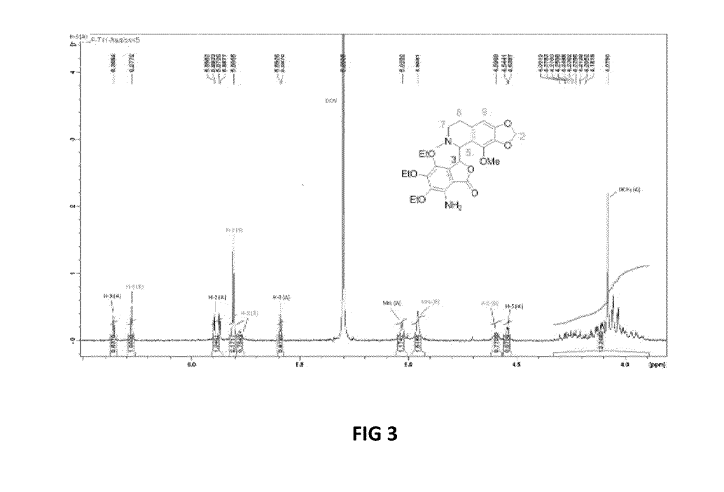

### DETAILED DESCRIPTION OF THE INVENTION

The tritoqualine salt synthesized with ethanoic acid (tritoqualine acetate) was prepared in the following manner:

### EXAMPLE 1

Ethanoic acid is added at a dose of 0.01 M (i.e. 6 g) into acetone (125 ml) and the same molar amount of tritoqualine base (0.01 M, i.e. 5 g) is added to the acetone.

This mixture is gradually heated and reduced in a vacuum evaporator until a precipitate is obtained. This precipitate is redissolved in tetrahydrofuran. A further precipitation is again performed. A white crystalline powder is obtained.

The dissolution tests show a very substantial and surprising increase in tritoqualine.

Tritoqualine acetate has a solubility of 0.75 M/L, i.e. an increase of 1500 times the initial solubility of tritoqualine in water.

### EXAMPLE 2

The tritoqualine salt synthesized with fumaric acid (tritoqualine fumarate) was prepared in the following manner:

Fumaric acid is added at a dose of 0.01 M (i.e. 1.16 g) to acetone (125 mL) and the same molar amount of tritoqualine base (0.01 M, i.e. 5 g) is added to the acetone.

This mixture is gradually heated from 30 to 75° and reduced in a vacuum evaporator until a precipitate is obtained. This precipitate is redissolved in tetrahydrofuran. A further precipitation is again performed. A white crystalline powder is obtained.

The dissolution tests show a very substantial and surprising increase in tritoqualine.

Tritoqualine fumarate has a solubility of 7.5 M/L, i.e. an increase of 15 000 times the initial solubility of tritoqualine in water.

### EXAMPLE 3

The tritoqualine salt synthesized with camphorsulfonic acid (tritoqualine camphorsulfonate) was prepared in the following manner:

Camphorsulfonic acid is added at a dose of 0.01 M (i.e. 2.32 g) to acetone (125 ml) and the same molar amount of tritoqualine base (0.01 M, i.e. 5 g) is added to the acetone.

This mixture is gradually heated from 30 to 75° and reduced in a vacuum evaporator until a precipitate is obtained. This precipitate is redissolved in tetrahydrofuran. A further precipitation is again performed. A slightly yellow crystalline powder is obtained.

The dissolution tests show a very substantial and surprising increase in tritoqualine.

Tritoqualine camphorsulfonate has a solubility of 5.5 M/L, i.e. an increase of 11 000 times the initial solubility of tritoqualine in water.

### EXAMPLE 4

The tritoqualine salt synthesized with phosphoric acid (tritoqualine phosphate) was prepared in the following manner:

Phosphoric acid is added at a dose of 0.01 M (i.e. 0.97 g) to acetone (125 ml) and the same molar amount of tritoqualine base (0.01 M, i.e. 5 g) is added to the acetone.

This mixture is gradually heated from 30 to 75° and reduced in a vacuum evaporator until a precipitate is obtained. This precipitate is redissolved in tetrahydrofuran. A further precipitation is again performed. A slightly straw-yellow crystalline powder is obtained.

The dissolution tests show a very substantial and surprising increase in tritoqualine.

Tritoqualine phosphate has a solubility of 15.5 M/L, i.e. an increase of 31 000 times the initial solubility of tritoqualine in water.

### EXAMPLE 5

The tritoqualine salt synthesized with sulfurous acid (tritoqualine sulfate) was prepared in the following manner:

Sulfurous acid in sulfite form is added at a dose of 0.01 M (i.e. 0.82 g) to acetone (125 ml) and the same molar amount of tritoqualine base (0.01 M, i.e. 5 g) is added to the acetone.

This mixture is gradually heated from 30 to 75° and reduced in a vacuum evaporator until a precipitate is obtained. This precipitate is redissolved in tetrahydrofuran. A further precipitation is again performed. A slightly pale yellow crystalline powder is obtained.

The dissolution tests show a very substantial and surprising increase in tritoqualine.

Tritoqualine sulfate has a solubility of 1.25 M/L, i.e. an increase of 2500 times the initial solubility of tritoqualine in water.

### EXAMPLE 6

The tritoqualine salt synthesized with methylsulfonic acid (tritoqualine methylsulfonate) was prepared in the following manner:

Methylsulfonic acid is added at a dose of 0.01 M (i.e. 0.96 g) to acetone (125 ml) and the same molar amount of tritoqualine base (0.01 M, i.e. 5 g) is added to the acetone.

This mixture is gradually heated from 30 to 75° and reduced in a vacuum evaporator until a precipitate is obtained. This precipitate is redissolved in tetrahydrofuran. A further precipitation is again performed. A slightly pale yellow crystalline powder is obtained.

The dissolution tests show a very substantial and surprising increase in tritoqualine.

Tritoqualine methylsulfonate has a solubility of 25 M/L, i.e. an increase of 50 000 times the initial solubility of tritoqualine in water.

### EXAMPLE 7

The tritoqualine salt synthesized with hydrochloric acid (tritoqualine hydrochloride) was prepared in the following manner:

Hydrochloric acid is added at a dose of 0.01 M (i.e. 0.96 g) to acetone (125 ml) and the same molar amount of tritoqualine base (0.01 M, i.e. 5 g) is added to the acetone.

This mixture is gradually heated from 30 to 75° and reduced in a vacuum evaporator until a precipitate is obtained. This precipitate is redissolved in tetrahydrofuran. A further precipitation is again performed. A white crystalline powder is obtained.

The dissolution tests show a very substantial and surprising increase in tritoqualine.

Tritoqualine hydrochloride has a solubility of 0.25 M/L, i.e. an increase of 500 times the initial solubility of tritoqualine in water.

1. Tritoqualine salt, having a structure formed from a base and an acid coupled ionically according to the following general formula.

2. Tritoqualine salt according to claim 1, wherein the base has a racemic form of tritoqualine.

3. Tritoqualine salt according to claim 1, wherein the base has a racemic form which is chosen from the R,R and S,S form.

4. Tritoqualine salt according to claim 1, wherein the coupled acid is chosen from the following acids: ethanoic acid, adipic acid, hexanedioic acid, o-aminobenzoic acid, anthranilic acid, m-aminobenzoic acid, para-aminobenzoic acid, glycine, ascorbic acid, aspartic acid, benzoic acid, bromoethanoic acid, o-bromobenzoic acid, m-bromobenzoic acid, citric acid, lactic acid, 2-hydroxypropanoic acid, maleic acid, z-butenedioic acid, malonic acid, propanedioic acid, hydrochloric acid, sulfurous acid, methylsulfonic acid, mesylate, camphorsulfonic acid, and also fumaric acid.

5. Method of improving the solubility of tritoqualine in bodily fluids and in aqueous media which comprises adding an effective amount of the tritoqualine salt of claim 1 to a bodily fluid or aqueous media.

6. Method of preparing injectable solutions, eye lotions, nasal solutions, or nebulizers which comprises adding an effective amount of the tritoqualine salt of claim 1 to the injectable solutions, eye lotions, nasal solutions, or nebulizers.

7. An injectable solution, eye lotion, nasal solution, or nebulizer comprising the tritoqualine salt of claim 1.
